(19) United States
(12) Patent Application Publication
(10) Pub. No.: US 2009/0035865 A1
(43) Pub. Date: Feb. 5, 2009

(54) MOISTURE SENSOR

(76) Inventors: Colette Pamela DeMoor, Palo Alto, CA (US); Ashok Sinha, San Jose, CA (US); Ian Gibbons, Portola Valley, CA (US)

Correspondence Address: DINSMORE & SHOHL, LLP;ONE DAYTON CENTRE, ONE SOUTH MAIN STREET, SUITE 1300, DAYTON, OH, 45402, US

(21) Appl. No.: 11/832,130

(22) Filed: Aug. 1, 2007

(51) Int. Cl. G01N 33/18; B01J 19/00; G01N 21/78

(52) U.S. Cl. 436/39; 422/55; 422/83

(57) ABSTRACT

A moisture sensor is disclosed which measures cumulative exposure to moisture. The moisture sensor comprises a matrix having a hygroscopic material, a first reagent, and a second reagent which interact only in the presence of water to produce a detectable, irreversible change in the matrix to provide a moisture indication. The moisture sensor may be incorporated with a disposable diagnostic device, whereby a method for preventing the use of such a disposable diagnostic device if exposed to excessive cumulative humidity is also disclosed.

### BACKGROUND OF THE INVENTION

The present invention relates generally to moisture sensors, and in particular, to moisture sensors which measure cumulative moisture exposure.

In diagnostic systems, using single-use cartridges containing reagents for carrying out sample assays (disposable diagnostic devices), assay calibration is established typically at the time of manufacture for a particular lot of cartridges. Calibration information is provided to the user in a variety of ways, for example by bar coding on the cartridge or as a bar-coded card that is “read” upon insertion of the cartridge into an instrument. A feature of this calibration scheme is that the cartridge should not change in any characteristic that would adversely affect the assay result between manufacture and use. Providing a defined lifetime for the particular lot in defined “worst-case” environmental conditions ensures that such characteristics of the cartridge do not change. The manufacturer then warrants the assay performance if the storage and handling of the cartridges has been less than or equally severe to these worst-case conditions.

In general, cumulative exposure to moisture in the atmosphere is one of the major contributors to the deterioration of a single-use assay cartridge. Accordingly, it would be useful to provide a moisture sensor that shows a record of the total exposure of the cartridge to unfavorable moisture conditions. In addition, reading such a moisture sensor should be easy, and incorporating such a sensor into a cartridge for use in a diagnostic assay system should be inexpensive.

### SUMMARY OF THE INVENTION

It is against the above background that the present invention provides a non-reversible moisture sensor which measures the cumulative exposure to moisture, and which can be easily read and inexpensively incorporated into a cartridge for use in a diagnostic assay system.

In one embodiment, the present invention provides a moisture sensor for detecting cumulative relative humidity. The moisture sensor comprises a matrix having a first reagent, and a second reagent, wherein the first and second reagents are capable of interacting in the presence of water to produce a detectable, irreversible change in the matrix but do not react in the same manner in the absence of water. The matrix includes a hygroscopic material capable of absorption of atmospheric moisture, wherein the absorption of atmospheric moisture by the hygroscopic material causes the interaction of the first and second reagents, thereby producing the detectable, irreversible change in the matrix.

In another embodiment, the present invention provides a method for preventing the use of a disposable diagnostic device which has been exposed to excessive humidity. The method comprises providing a moisture sensor to the disposable diagnostic device, wherein the moisture sensor comprises a layered matrix having a first reagent, and a second reagent, wherein the first and second reagents are capable of interacting to produce a detectable, irreversible change in the matrix. The sensor further includes a hygroscopic material capable of absorption of atmospheric moisture, wherein the absorption of atmospheric moisture by the hygroscopic material causes the interaction of the first and second reagents, thereby producing the detectable, irreversible change in the matrix. The method further comprises determining the cumulative moisture exposure indicated by the sensor; and excluding from analysis any of the devices having a cumulative moisture exposure in excess of a predetermined amount.

These and other features and advantages of the invention will be more fully understood from the following description of various embodiments of the invention taken together with the accompanying drawings.

### BRIEF DESCRIPTION OF THE DRAWINGS

The following detailed description of the embodiments of the present invention can be best understood when read in conjunction with the following drawings, where like structure is indicated with like reference numerals, and in which:

FIG. 1 is a block diagram of an illustrated embodiment of a moisture sensor having a matrix according to the present invention used with a diagnostic device, and optionally an electronic instrument;

FIGS. 1A-1L are block diagrams of various embodiments of arrangements of providing the reagents and hydroscopic material that comprise the matrix of FIG. 1;

FIG. 2 is a plot showing the effect of ferric sulfate concentration on the rate of color production by transmissive sensors according to the present invention;

FIG. 3 is a plot showing the effect of moisture on the rate of color formation by a transmissive sensor according to the present invention;

FIG. 4 is a plot showing the effect of moisture on rate of color formation for reflective sensors according to the present invention;

FIG. 5 is a plot showing the effect of varying “filler” components on rate of color formation by transmissive sensors according to the present invention;

FIG. 6 is a plot showing a comparison of transmissive sensors containing gelatin and a non-reducing sugar to sensors without gelatin on rate of color formation according to the present invention;

FIG. 7 is a plot showing the effect of varying gelatin type on rate of color formation of transmissive sensors according the present invention;

FIG. 8 is a plot showing the effect of drying temperature on rate of color formation of transmissive sensors according the present invention;

FIG. 9 is a plot showing the effect of drying time on rate of color formation of transmissive sensors (average readings from three sensors) according to the present invention;

FIG. 10 is a plot showing the effect of storage time on the rate of color formation of transmissive sensors (average absorbance values from triplicate readings) according to the present invention;

FIG. 11 is a plot showing the effect of storage temperature on color formation of transmissive sensors according to the present invention;

FIG. 12 is a plot showing color change at room humidity (about 45% RET) of a reflective moisture sensors based on NaOH and cresol red according to the present invention; and

FIGS. 13 and 14 are plots showing a comparison of transmissive sensors according to the present invention located in either an opened cartridge or a diffusion channel in a cartridge at different humidities.

Skilled artisans appreciate that elements in the figures are illustrated for simplicity and clarity and have not necessarily been drawn to scale or in a particular orientation. For example, the dimensions of some of the elements in the figures may be exaggerated relative to other elements, and with conventional parts removed, to help to improve understanding of embodiments of the present invention.

### DETAILED DESCRIPTION

As disclosed herein, the present invention provides various moisture sensor embodiments that permanently records exposure to atmosphere moisture. Each of the various embodiments disclosed herein either indicates the cumulative amount of atmospheric moisture to which the sensors have been exposed or indicates cumulative exposure to atmospheric moisture at a level above a predetermined value. The moisture sensors are easily readable visibly either by a human or machine. As will be explained herein, the present invention allows the user to determine if a disposable diagnostic device equipped with a moisture sensor has been exposed to moisture conditions outside the range of warranted conditions.

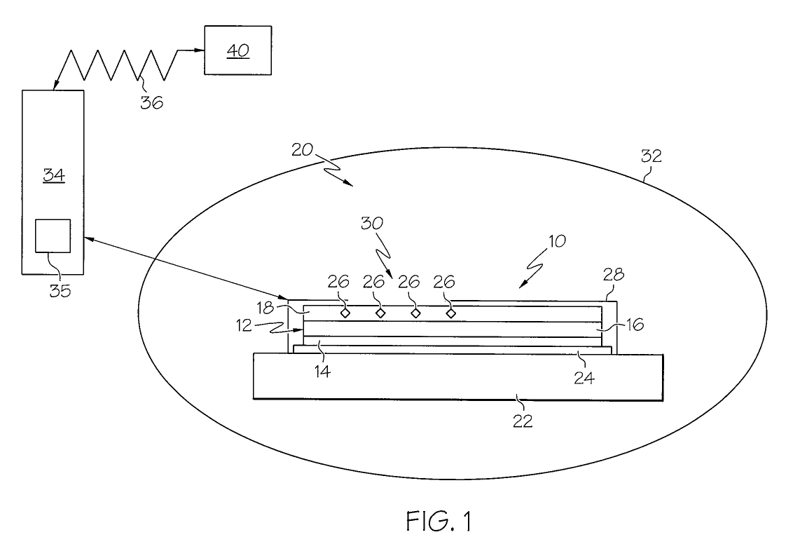

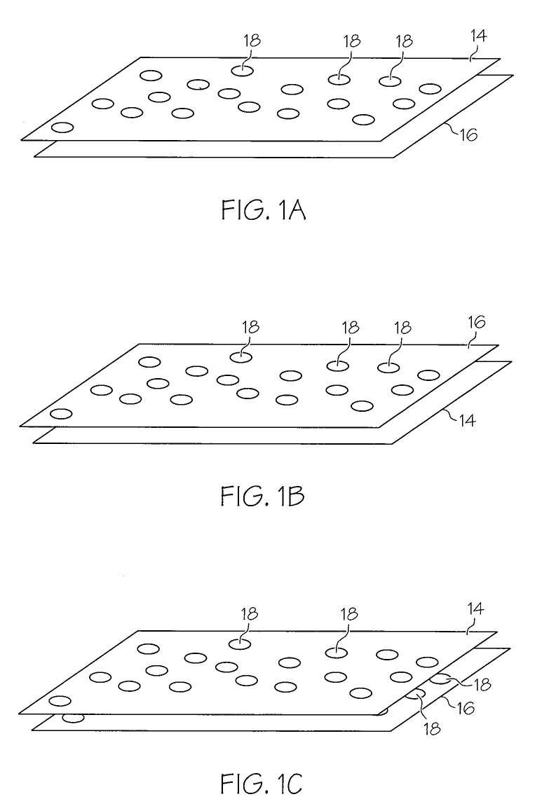

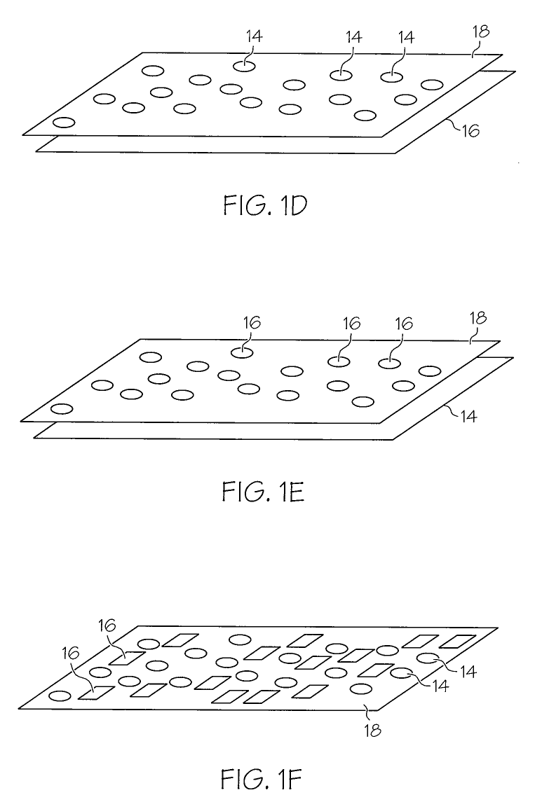

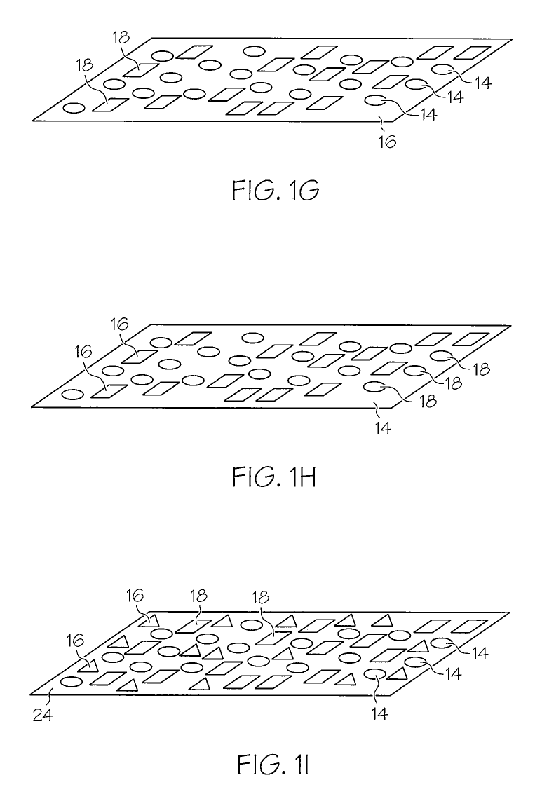

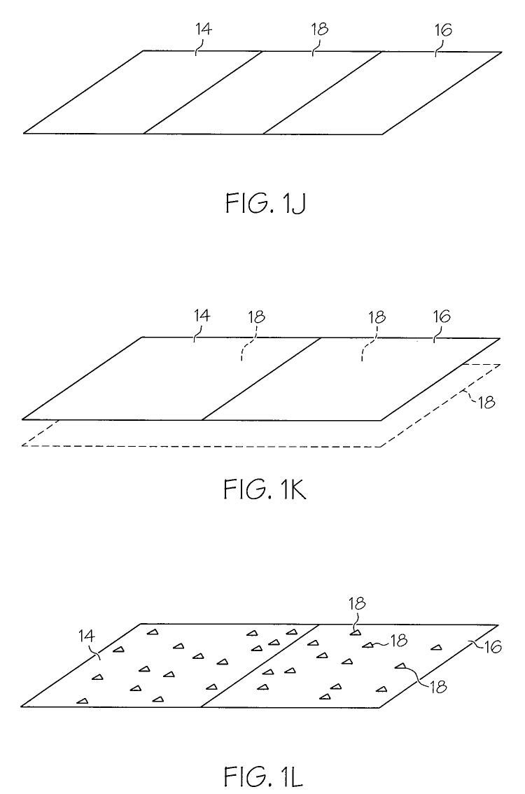

In various embodiments, the sensor is useful for indicating the cumulative moisture exposure of disposable diagnostic devices containing analytical reagents sensitive to a known cumulative moisture exposure that would impair their use. In one embodiment, the sensor would start to provide a color change at a cumulative moisture exposure less than that of the known cumulative moisture exposure. In one embodiment, a particular color indicates that the known cumulative moisture exposure level has been reached, such as for example, a red display. Optionally, an analytical instrument used in connection with the disposable diagnostic device can automatically read the sensor, via an optical sensor, and reject the associated disposable diagnostic device if defective without user intervention. With these various embodiments in mind, attention is directed to FIG. 1.

FIG. 1 is an illustrated embodiment of a moisture sensor according to the present invention, which is generally indicated by symbol 10. The sensor 10 in one embodiment comprises a matrix 12 having a first reagent 14, a second reagent 16, and a hygroscopic material 18. FIGS. 1A-1L are illustrative examples of a few suitable arrangements of the first reagent 14, the second reagent 16, and the hydroscopic material 18 which comprise the matrix 12 according to the present invention. However it is to be appreciated that the hygroscopic material 18 can be the first reagent 14, the second reagent 16, or it can be a separate compound. Thus, a reference herein to “a first reagent, a second reagent, and a hygroscopic material” should not be interpreted as requiring three separate compounds, although such is the case in most of the illustrated embodiments. Typically, the moisture sensor 10 is formed and maintained in a completely anhydrous environment until used.

In the illustrated embodiment of FIG. 1, the matrix 12 is formed from two or more connected layers in which the first reagent 14 and the second reagent 16 are distributed in different layers. In this embodiment, the hygroscopic material 18 is provided in a third layer. In an alternative embodiment, such as illustrated by FIG. 1A, the hydroscopic material 18 is interspersed throughout a layer of the first reagent 14, which is provide on a layer of the second reagent 16.

In other embodiments, the hydroscopic material 18 may be distributed only to the layer of the second reagent 16 or to both layers, such as illustrated by FIGS. 1B and 1C, respectively. In still other embodiments, either or both reagents 14, 16 can be interspersed throughout a layer of the hydroscopic material 18, or vice-versa, such as illustrated by FIGS. 1D-1H. In still another embodiments, the reagents 14, 16 and the hydroscopic material 18 is interspersed throughout a support layer 24, such as illustrated by FIG. 1I. In other embodiments, the matrix 12 is formed as a single homogeneous layer containing the first reagent 14, the second reagent 16, and the hygroscopic material 18 such as illustrated by FIG. 1J. In still other embodiment, the hydroscopic material 18 may or may not be different from either the first or the second reagent 14, 16, such as illustrated by FIG. 1K via the dashed lines.

In still other embodiments, the matrix 12 can be formed as a discontinuous and/or inhomogeneous layer of the first and second reagent 14, 16 which is interspersed with the hydroscopic material 18 in an unequal fashion throughout the layer, as illustrated by FIG. 1L. The particular combination and orientation of the layers in the matrix 12, and the distribution and quantities of the reagents 14, 16 and the hygroscopic material 18, will depend largely upon the particular reagents and the hygroscopic material employed. Thus, although this specification often refers to the matrix 12 as “layered,” which in some embodiment may be so, the word layered as used in other embodiments may just indicate that the two reagents 14 and 16 are present in the matrix in a form in which they do not react with each other in the absence of moisture (i.e., water).

In use, the sensor 10 is activated when the hygroscopic material 18 absorbs atmospheric moisture from an ambient environment, indicated generally by arrows 20, and the presence of sufficient moisture absorbed by the hygroscopic material 18 causes an interaction between the first and second reagents 14 and 16 to produce a detectable, irreversible change in the matrix 12. It is to be appreciated that the first and second reagents 14 and 16 are selected such that they do not react in the same manner either in the absence of water or individually in the presence of water.

The moisture sensor 10 can be affixed to, or made an integrated part of, a disposable diagnostic device 22 to provide an easily readable indication of the moisture exposure of the moisture-sensitive components of the device. A support 24 and an adhesive (not shown) may be provided to help affix the sensor 10 to a desired surface, such as the disposable diagnostic device 22. A variety of disposable diagnostic devices with which the present invention can be used are disclosed in U.S. Pat. Nos. 4,756,884, D302,294, 4,753,776, 4,868,129, 5,077,017, 5,028,142, 5,039,617, 5,104,813, 4,952,373, 5,230,866, and 5,279,791, which disclosures are incorporated herein by reference.

In the illustrated embodiment of FIG. 1, water is brought into the sensor 10 by the hygroscopic material 18. The reagents 14 and 16 act as if they are in separate “layers” and diffuse between the “layers” in the presence of water. As with the reagents 14 and 16, the hygroscopic material 18 can be distributed homogeneously throughout the matrix 12, or can be interspersed discontinuously.

The first and second reagents 14 and 16 will be substances capable of interacting, either directly or indirectly, to produce a detectable, irreversible change in the matrix 12. By irreversible it is meant that, once accomplished, the change is not reversed for the useful life of the sensor. Depending upon the particular application, the useful life of the sensor can range from several hours to several decades.

Interacting directly means that the first and second reagents 14 and 16 themselves interact to produce a detectable, irreversible change in the matrix 12. Interacting indirectly means that the first reagent 14 (or the second reagent 16) forms a distinct product or products due to absorption of sufficient moisture by the hygroscopic material 18. The resulting distinct product (or products) then interacts with the second reagent 16 (or the first reagent 14), or a product of the second reagent 16 (or a product of the first reagent 14), to produce a detectable irreversible change in the matrix 12. An example of an indirect interaction is one in which the first reagent 14 is an enzyme substrate which in the presence of sufficient moisture absorbed by the hygroscopic material 18 forms a product which interacts then with the second reagent 16 to produce a detectable, irreversible change in the matrix 12.

An irreversible change will be one which alters the optical properties of the matrix 12, for example, as when a colorless matrix becomes colored, or a transparent matrix becomes opaque, or vice versa. Changes in color or opacity (or degrees thereof) can be determined by monitoring the optical transmittance or reflectance of the matrix. As used herein, a transmissive sensor is one that is designed to be monitored by measurement of its transmissive properties; and a reflective sensor is designed to be monitored by measurement of its reflective properties.

In one embodiment, the first and second reagents 14 and 16 are chemical or biological substances which interact to produce a reaction product or products having optical properties different from those of the first and second reagents. Examples of chemical reagents are colorless (or slightly colored) inorganic or organometallic salts or organic compounds which interact with previously immobile ligands that become mobile in the presence of water to form colored complexes, as well as, colored complexes that similarly react with ligands to produce complexes of different color. The moisture-mobilized ligands are selected so that the resulting complex is more stable than the original complex, thereby resulting in an irreversible change.

In one embodiment, the first reagent 14 is an acid or base and the second reagent 16 is a color change indicator, such as for example an acid-base indicator or a pH sensitive dye, which together react in the presence of moisture to produce a color change in the matrix 12. It will be obvious that the first and second reagents 14 and 16 are interrelated in that the possible second reagent 16 is determined by the choice of the first reagent 14. In one embodiment, interaction between iron (III) salts and thiocyanate salts produce the highly colored iron (III) thiocyanate complex. For this embodiment, suitable first reagents include a number of iron (III) salts, including ferric sulfate, particularly the pentahydrate, ferric nitrate, particularly the nonahydrate, and ferric chloride, particularly the hexahydrate. Accordingly, for the above embodiment, suitable second reagents include thiocyanate salts, including ammonium thiocyanate, lithium thiocyanate, sodium thiocyanate, potassium thiocyanate, guanidine thiocyanate, tetrabutylammonium thiocyanate, and the like.

In another embodiment, a combination of first and second reagents 14 and 16 is ferric sulfate and tetrabutylammonium thiocyanate. In still other embodiments, other metal salts and metal chelating agents that produce colored complexes can be used. Salts of iron, cobalt, nickel, chromium, manganese or copper as the first reagent 14 can be combined with a second reagent 16 comprising a thiocyanate compound, a ferrocyanide (particularly with iron (III) salts), alpha-nitrosonaphthol (particularly with cobalt salts) or chrome azurol S.

Other embodiments having combinations of the first and second reagents 14 and 16 include a number of acids or bases as first reagent, particularly sodium hydroxide or potassium hydroxide, and a number of acid-base indicator dyes as second reagent, particularly cresol red. Other combinations include acids such as oxalic acid or lactic acid as the first reagent 14 and acid-base indicators such as thymol blue, tropaeolin 00, methyl yellow, methyl orange, bromocresol green, methyl red, bromothymol blue, phenol red, phenolphthalein, or thymolphthalein as the second reagent 16. Any combination can be used that provides the desired color or opacity change.

In still other embodiments, the first and second reagents 14 and 16 are biological substances, for example an enzyme, and a chromogenic substrate of the enzyme. In alternative embodiments, either the first reagent 14 or the second reagent 16 can be generated in situ, for example, by the action of an enzyme. An example of this embodiment is the combination of oxidase-peroxidase enzymes and the oxidizable chromogens DAOS(N-ethyl-N-(2-hydroxy-3-sulfopropyl)-3,5-dimethoxyaniline) (e.g., a substituted aniline) and aminoantipyrine. This pair of chromogens gives a colored product when hydrogen peroxide is formed by the enzymatic conversion of glucose (or cholesterol) by the oxidase and atmospheric oxygen. The color-making reaction is catalyzed by the peroxidase. When dry, the reagents 14 and 16 remain colorless. On exposure to moist air, however, color is formed resulting in a change in opacity, presumably due to reaction with oxygen in the presence of moisture.

The hygroscopic material 18 will be any compound capable of absorbing moisture 20 from the surrounding atmosphere. In one embodiment, ferric chloride and ammonium thiocyanate are hygroscopic materials which are also used as the first and second reagents 14 and 16. In other embodiments, suitable hygroscopic materials include calcium chloride, cobalt chloride, zinc chloride, zinc bromide, sodium hydroxide or any commonly used drying agent. An alkyl halide quanternary salt of an alkyl methacrylate may also be used in other embodiments. As with first and second reagents 14 and 16, the choice of hygroscopic material 18 may be limited by the choice of first and second reagents. The hygroscopic material 18 will normally be selected so as not to interfere in an adverse manner with the interaction between the first and second reagents 14 and 16.

The layer or layers forming the matrix 12 can be a dried film or a “glass” produced by the use of a forming agent, such as a film-forming or glass-forming agent, in combination with one or more of the reagents 14 and 16. Such glass-forming agents are well known in the art and examples include sugars such as sucrose, trehalose, and mannitol. Film-forming agents include proteins such as gelatin, and polymers such as polyvinylpyrrolidone (PVP-10), polyethylene glycol (PEG 15,000-20,000 MW), hydroxypropylmethyl cellulose, and cellulose ether. In various embodiments, the forming agents are ones which form clear, colorless glasses or films, and can include clear films that adhere to substrates such as plastic or glass. In other embodiments, such as those in which a change in reflectance property of the matrix is measured, an initial clear colorless glass or film is not needed; therefore, other glass-forming or film-forming agents can be used. In some embodiments, the layers can be formed without the aid of any forming agent, but instead can be formed by evaporating the solvent from a solution of appropriate components as described in more detail below.

The layer(s) of the matrix 12 can be formed directly on the surface of the diagnostic device 22. In an alternative embodiment, the layer(s) of the matrix 12 can be formed optionally on the support 24, which may be a material, such as for example, paper, cloth, felt, or porous membranes. Other support material, such as glass or plastic, can also be used, if the material used does not prevent the interaction between the first and second reagents 14 and 16, for example, by preventing the access of the reagents to the moisture 20 absorbed by the hygroscopic material 18. In some embodiments, the support 24 can provide the means for controlling the rate of the interaction of first and second reagents 14 and 16 and, as such, can inhibit the reaction by limiting access to atmospheric moisture 20. When a support 24 is used, one embodiment of a method of construction of the sensor 10 uses a solution containing some combination of first reagent 14, second reagent 16, and hygroscopic material 18 to coat the support 24. This coating is then dried. Any remaining components can be coated separately onto the same support or onto additional supports. If more than one layer of the support 24 is used, the layers can be calendered together to form a multi-layered matrix 12.

The illustrative embodiments, containing separate reagent layers 14 and 16, are constructed by spreading a first layer of an aqueous solution of ferric sulfate (first reagent 14) containing a film-forming agent onto a support 24 and allowing the layer to dry. A second layer of tetrabutylammonium thiocyanate (second reagent 16) dissolved in an organic solvent is then spread on the first layer in a dry environment. The organic solvent is chosen such that the ferric sulfate is insoluble and, therefore, the ferric sulfate and the tetrabutylammonium thiocyanate reagents 14 and 16 remain in separate layers. The organic solvent is evaporated, resulting in a two-layered matrix having a separate ferric sulfate layer and tetrabutylammonium thiocyanate layer. The matrix 12 is clear and slightly yellowish in color. In one embodiment, organic solvents include dichloromethane and dimethoxymethane. In one embodiment, a hygroscopic material 18 may be provided on top of the second layer 16 to control the rate of interaction, which is discussed greater in a later section, and in other embodiments, ferric sulfate may act as the hygroscopic material. When sufficient moisture is absorbed by the hygroscopic material 18 (or the ferric sulfate if the hygroscopic material), the reaction between the ferric sulfate and the tetrabutylammonium thiocyanate occurs, producing the darkly colored, red ferric thiocyanate complex.

To be useful as an indicator of the cumulative exposure to moisture of the diagnostic device 22 containing moisture-sensitive components, the response of the moisture sensor 10 should anticipate the kinetics of the deterioration of the moisture-sensitive components of the diagnostic device 22. For example, under exposure conditions which cause the moisture-sensitive components to begin to deteriorate, the moisture sensor 10 must clearly indicate excessive exposure. In other words, the moisture sensor 10 should be at least as sensitive to moisture as the moisture-sensitive components of the diagnostic device 22.

For most purposes, the moisture sensor 10 is constructed to record any excessive moisture exposure. In most embodiments, the moisture-sensitive components will be biological or chemical reagents necessary to carry out the assay for which the diagnostic device 22 was designed, but the moisture-sensitive components can also be any other component of the diagnostic device which is subject to deterioration because of exposure to excessive moisture. By excessive moisture, it is meant that relative humidity conditions are higher than those for which the diagnostic device 22 is warranted.

The appropriate sensitivity of the moisture sensor 10 can be achieved in a number of ways. In some embodiments, the kinetics of the interaction between the first reagent 14 and the second reagent 16 in the presence moisture may fortuitously be similar to the kinetics of deterioration of the moisture-sensitive components of the diagnostic device 22. Additional control of the rate of interaction can be obtained by adjusting the concentrations of the first reagent 14, second reagent 16, and/or hygroscopic material 18 in the matrix 12 or by adding an additional rate control substance 26 which inhibits (reduces) or enhances (increases) the interaction. Such a rate-controlling substance can be materials such as non-reducing sugars, or natural or synthetic polymers such as gelatin or polyvinylpyrrolidone (PVP).

In still other embodiments, the rate of interaction of the first reagent 14 and second reagent 16 (and hence the sensitivity of the sensor 10) can be controlled by physical means, such as by providing a physical barrier 28 between the first reagent 14 and the second reagent 16, between either or both reagents 14, 16 and the hygroscopic material 18, or between the hygroscopic material 18 and the moisture 20 in the atmosphere. Examples of such physical barriers 28 are semi-permeable membranes, such as microporous polytetrafluoroethylene, which can be placed between the first and second reagents 14 and 16, between the reagents 14 and 16 and the hygroscopic material 18, or between the hygroscopic material 18 and the ambient environment. In the illustrated embodiment, the physical barrier 28 encloses the sensor 10 to control exposure of the sensor to the ambient (external) environment. In such an embodiment, diffusion of atmospheric moisture 20 to the sensor 10 is controlled by providing a narrow diffusion channel 30 between the environment and the hygroscopic material 18 of the sensor 10.

In one embodiment, the sensor 10 is incorporated within the disposable diagnostic device 22 such that its sole exposure to the surrounding atmosphere is through a diffusion channel 30. The rate of diffusion of atmospheric moisture 20 through the diffusion channel 30 can be controlled by changing the shape, length, and/or cross-sectional area of the diffusion channel 30. For example, the sensor 10 can be completely enclosed in a moisture-impervious housing (i.e., physical barrier 28) having a diffusion channel 30, such as for example, and not to be limited, 1-2 cm in length and having a cross-sectional area of 0.1 mm². The physical dimensions of the housing 28 and the desired rate of diffusion impose the only constraints on the length and cross-sectional area. In the illustrated embodiment, the rate of interaction of first and second reagents 14 and 16 is controlled by the rate of absorption of moisture by the hygroscopic material 18, which is in turn controlled by the rate of diffusion of the atmospheric moisture to the sensor 10, which in turn is controlled by the length and cross-sectional area of the diffusion channel 30.

The change in the matrix 12 of the moisture sensor 10 in response to sufficient moisture absorbed by the hygroscopic material 18 will be readily determined by any of a variety of methods, the particular method depending on the sensor used. In the simplest method, the change is a visually observable one which is determined by the user of the diagnostic device 22. The user will discard any diagnostic devices 22 in which the sensor 10 indicates an exposure to excessive moisture. In this case, the sensor 10 need not be affixed to an integral part of the diagnostic device 22 but need only be exposed to the same environmental conditions experienced by the diagnostic device 22. For example, the sensor 10 can be provided separately in package or container 32 also containing the device 22.

In another embodiment, an electronic instrument 34 used in connection with the diagnostic device 22 is configured to read the sensor 10, via an optical sensor 35, and determine the change in the matrix 12 of the moisture sensor 10 automatically, for example. In this case, the sensor 10 will generally be an integral part of, or permanently affixed to, the diagnostic device 22. In this embodiment, the electronic instrument 34 is used in connection with the diagnostic device 22 and is configured to prevent a user from obtaining diagnostic information from a diagnostic device 22 having a moisture sensor 10 which indicates exposure to excessive moisture, such as by displaying an error message and asking for insertion of another diagnostic device 22. In other embodiments, the electronic instrument 34 is configured to provide additional error reporting through a network 36 (wired or wireless) providing the lot number or other identification number for the diagnostic device 22 when an excess exposure to moisture is detected by the moisture sensor 10, thereby rendering the diagnostic device unusable. Such additional error reporting may be provided to a system/device 40 of a custodian of the diagnostic device and/or manufacturer, such that corrective procedures may be taken. For example, if a number of diagnostic devices 22 at different sites are indicating exposure to excessive moisture, such may be an indication that a particular lot needs a general recall due to possibly packaging, shipping, and/or handling problems of the associated containers 32.

The moisture sensor 10 can be affixed to the disposable diagnostic device 22 by methods that are well known in the art. The layered matrix 12 of the moisture sensor 10 can be constructed directly positioned on the diagnostic device 22, particularly when no other support is used. When a support 24 is used, the moisture sensor 10 can be affixed to the diagnostic device 22 using any appropriate adhesive or bonding compound. In an alternative embodiment, the moisture sensor 10 can be formed as an integral part of the diagnostic device 22 using the techniques of device production given in the previously listed patents and applications that have been incorporated by reference.

Use of the moisture sensor 10 to record cumulative exposure to excessive moisture is not limited to diagnostic devices 22 or instrumentation. The moisture sensor 10 can be used with and affixed to any device, room, chamber, instrument, or substance that is moisture sensitive. If the moisture sensor 10 cannot be attached to a device, instrument, or substance, the sensor can be located adjacent to the moisture sensitive object. Adjacent refers to a location of the moisture sensor 10, close enough to the moisture sensitive object to detect excessive moisture exposure of the object. The following explicit embodiments are provided by way of illustration and not by way of limitation.

For a first explicit embodiment, a ferric sulfate-tetrabutylammonium thiocyanate based transmissive sensor was prepared. It is to be appreciated that the below mentioned concentrations of mannitol, gelatin, ferric sulfate, tetrabutylammonium thiocyanate, and other components can be adjusted as appropriate. A first layer of ferric sulfate was prepared. A solution of 5% mannitol and 5% gelatin (by weight) was made in distilled water. This was heated slightly in order to dissolve the solutes. Fe₂(SO₄)₃.5H₂O, 25% by weight, was added to the mannitol/gelatin solution. This was again heated slightly to dissolve the ferric sulfate. The solution was sonicated for approximately 5 minutes to remove dissolved gases. Ten μl of the prepared ferric sulfate solution was dispensed on a plastic substrate. In order to obtain even films, the ferric sulfate solution was placed on the substrate and then spread evenly. The layer was dried at 37° C. at less than 5% relative humidity (RH) for 30 minutes. The drying temperature and drying time can be varied to achieve the desired dryness.

Next, a second layer of thiocyanate was prepared. A 50% by weight solution of tetrabutylammonium thiocyanate in dichloromethane (denoted tB/DCM) was prepared. Using a guide, 10 μl of the tB/DCM solution was added to the dried ferric sulfate layer in a dry environment. This was added as a drop and not spread evenly over the layer. The drop of tB/DCM was dried at 37° C. for 15 minutes to remove excess dichloromethane. Sensors prepared in the above manner, were then each stored in water impermeable containers containing silica gel. The containers were placed in a larger container containing a desiccant (i.e., anhydrous calcium sulfate), and then stored in the refrigerator.

In a second explicit embodiment, a method of testing the response of transmissive sensors according to the present invention to atmospheric moisture is disclosed. In this explicit embodiment, the formation of deep red color in response to environmental moisture was monitored by placing the sensors in environments of constant humidity and measuring the rate of color formation spectrophotometrically. Experiments were conducted in both open and closed desiccators. The closed desiccators contained either water, or salts which, when saturated in water, produce environments of known humidity. The open desiccators were used for experiments conducted at room humidity.

All the experiments were conducted at room temperature which remained constant at about 23° C. Three environments were considered: room humidity, extremely high humidity and above average humidity. Room humidity measurements ranged between 38% and 50% depending on the external weather conditions. The very high humidity environment was generated in a desiccator containing water. This produced a measured relative humidity of 88±2%. The humidity at which most of the experiments were conducted was the “above average” humidity environment, which was generated by placing a saturated calcium chloride solution in a desiccator. This produced an environment with a preserved relative humidity of 60±5%. Humidity measurements were made using a thermohygrometer, model number 8564 purchased from Hanna Instruments. Formation of ferric thiocyanate in response to humidity was determined by measuring the absorbance level at 480 nm of the sensor using a Hewlett Packard 8451 A Diode Array spectrophotometer.

Before each experiment, the sensors to be tested were removed from storage and allowed to equilibrate to room temperature in a desiccator containing a desiccant (i.e., anhydrous calcium sulfate) for approximately thirty minutes. The baseline absorbance was measured at 480 nm. Sensors were placed in one of the constant humidity environments. The rate of color formation was monitored every 10 to 15 minutes by measuring absorbance at 480 nm.

In a third explicit embodiment, a ferric sulfate-tetrabutylammonium thiocyanate based reflective sensor was prepared. The reflective sensors according to the present invention were prepared by dipping Whatman #1 filter paper in a 25% by weight solution of ferric sulfate and drying for 30 minutes using dry air at 37° C., and then quickly dipping the paper in tB/DCM before drying at 37° C. for 15 minutes. These reflective sensors were read by measuring reflectance on a Hewlett Packard 8452A Diode Array spectrophotometer fitted with a “Spectralon” reflectance attachment.

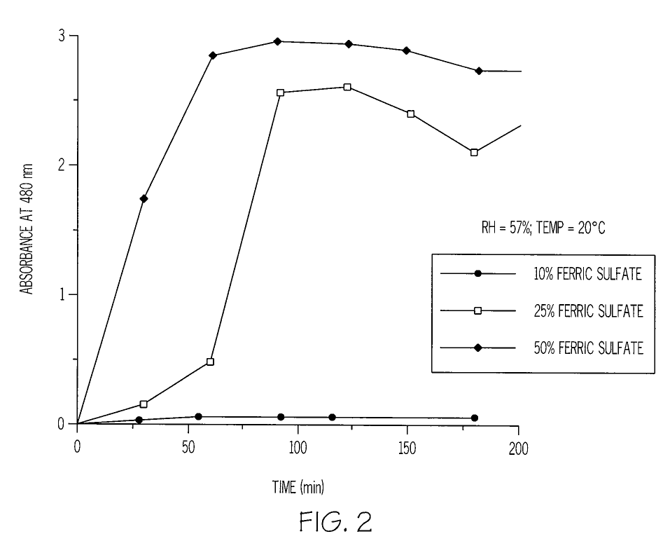

In a fourth explicit embodiment, the effect of a ferric sulfate concentration on the response of a transmissive sensor made as in the first explicit embodiment is disclosed. The effect of changing the ferric sulfate concentration on the rate of ferric thiocyanate formation in transmissive sensors is shown in FIG. 2. Sensors were made containing either 10%, 25%, or 50% ferric sulfate (w/v). The ferric sulfate layer also contained 7% gelatin to produce an even film. The second layer was formed from 4 μl of 20% tetrabutylammonium thiocyanate initially dissolved in dichloroethane, and then dried. The sensors were placed in 57% relative humidity and 20° C. Color formation was measured by monitoring absorbance at 480 nm. At absorbance levels above 2.0, the sensor is dark red. From FIG. 2 it can be seen that the sensors containing 50% ferric sulfate react faster, turning a dark red within 40 minutes as opposed to 90 minutes for those containing only 25% ferric sulfate. Sensors containing only 10% ferric sulfate did not react at all during the study. It is apparent from the results that changing the concentration of ferric sulfate in the iron layer can modify the kinetics of color formation.

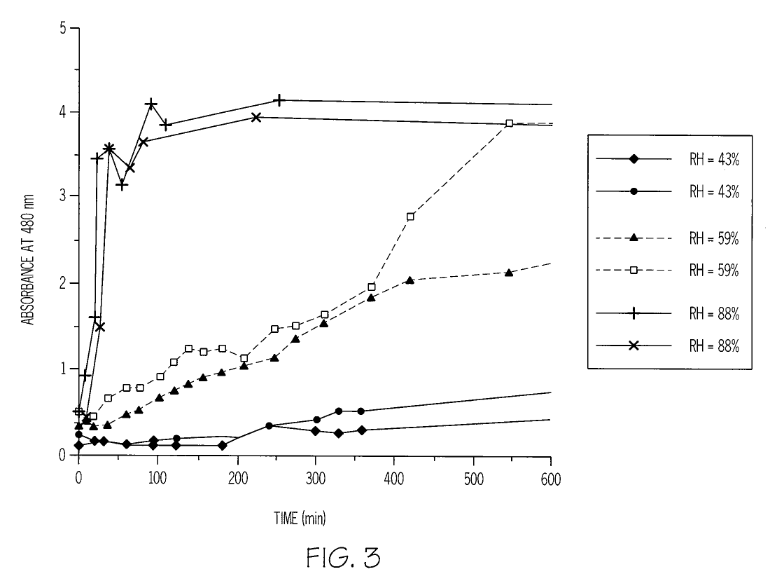

In a fifth explicit embodiment, a transmissive sensor response as a function of relative humidity is disclosed. FIG. 3 shows the effect of changing the relative humidity on the rate of color formation for transmissive sensors (made as in the first explicit embodiment). Duplicate sensors (made as in the first explicit embodiment) contained 25% ferric sulfate, 5% gelatin, and 5% mannitol in the ferric sulfate (first) layer and 50% tetrabutylammonium thiocyanate/dichloromethane in the thiocyanate (second) layer. The sensors were then placed in environments kept at relative humidities of 43% (room), 59%, or 88%. Color formation was measured as absorbance at 480 nm. At higher relative humidities, the rate of color formation is faster than at lower humidities. At 88% relative humidity, the red color is formed within 20 minutes, whereas at 59% it takes about 400 minutes for the same amount of color to form.

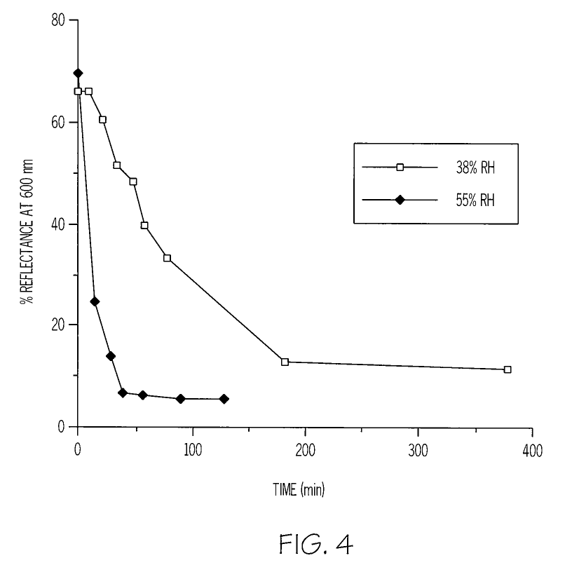

In a sixth explicit embodiment, reflective humidity sensors were made, as in the third explicit embodiment, by impregnating paper with an aqueous solution of 25% ferric sulfate and 15% mannitol, air-drying, and then reimpregnating with 50% tetrabutylammonium thiocyanate in dichloromethane (w/v), and drying again at 37° C. The sensors were then exposed to various humidities. The reflectance was measured at 600 nm and plotted as a function of time for samples at 38% (room) humidity, and at 55% relative humidity. This data is shown in FIG. 4. The reflective devices reacted faster than the transmissive devices of the fifth explicit embodiment, with color forming within 30 minutes for the sample in the higher humidity environment and within 200 minutes at room humidity.

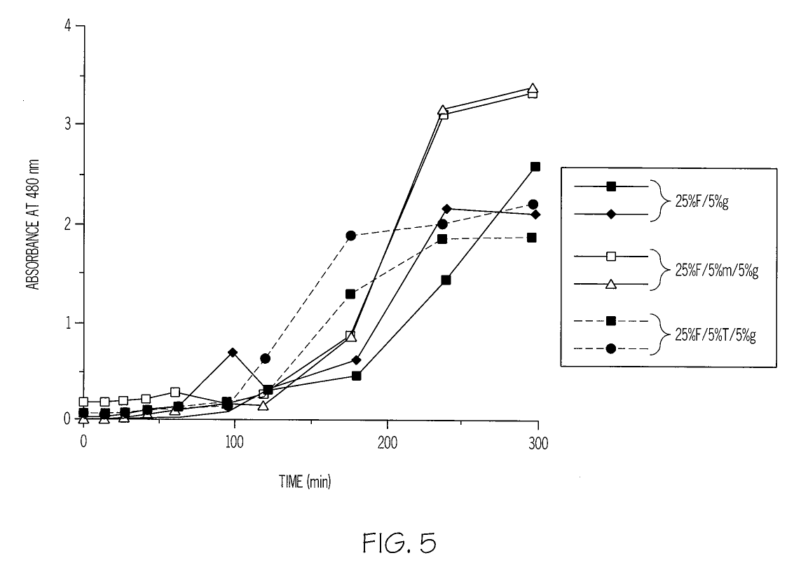

In a seventh explicit embodiment, the effect of film-forming components on transmissive sensor response is disclosed. The effect of changing the film-forming components on the kinetics and reproducibility of color formation is shown in FIGS. 5-7. The sensors were prepared as in the first explicit embodiment. In all these sensors, ferric sulfate was kept at 25% and the thiocyanate layer consisted of a 50% solution of tetrabutylammonium thiocyanate in dichloromethane. In addition, gelatin, (if used) was at 5% (w/v). Due to solubility problems, maximum concentrations of 5% mannitol or trehalose could be used.

For the results shown in FIG. 5, duplicate sensors were made having a first layer of 25% ferric sulfate and either 5% gelatin, 5% mannitol and 5% gelatin, or 5% trehalose and 5% gelatin (w/v). A second layer comprised 50% tetrabutylammonium thiocyanate in dichloromethane (w/v). These sensors were placed in an environment at 53% relative humidity and the rate of color formation was measured spectrophotometrically for 5 hours. Absorbance was measured at 480 nm.

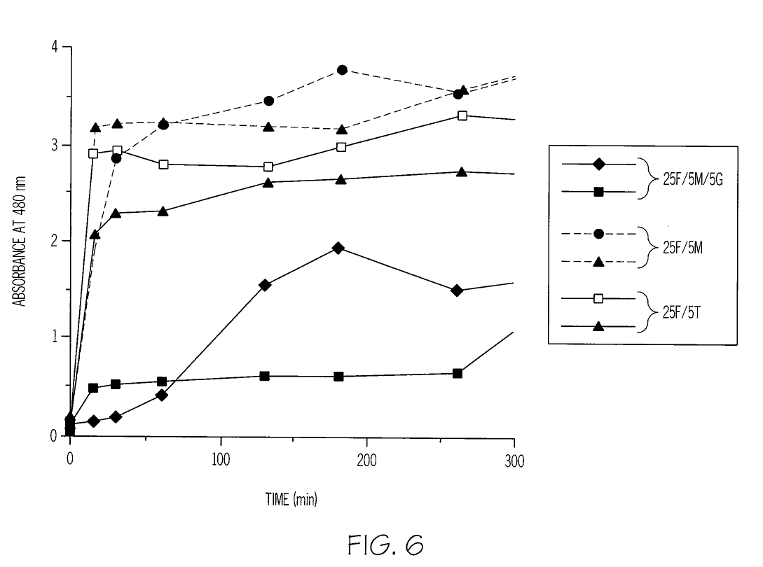

For the results shown in FIG. 6, duplicate sensors containing in a first layer 25% ferric sulfate and either 5% gelatin and 5% mannitol, 5% mannitol, or 5% trehalose. A second layer comprised 50% tetrabutylammonium thiocyanate in dichloromethane (w/v). These sensors were placed in an environment at 59% relative humidity and the rate of color formation measured for 5 hours. Absorbance was measured at 480 nm.

The sensors containing gelatin, shown in FIG. 5, exhibit a lag time before enough moisture is absorbed to generate color. Gelatin facilitates making clear films, and sensors containing gelatin have a longer shelf life than those not containing gelatin. Sensors containing no gelatin, shown in FIG. 6, however, have much faster and more reproducible kinetics than those containing gelatin. Due to the good reproducibility of the mannitol/gelatin sensor seen in FIG. 5, sensors with 25% ferric sulfate, 5% mannitol, and 5% gelatin in the ferric sulfate layer were used in the remaining explicit embodiments. Mannitol was also chosen over trehalose because sensors containing mannitol generated more color than those containing trehalose.

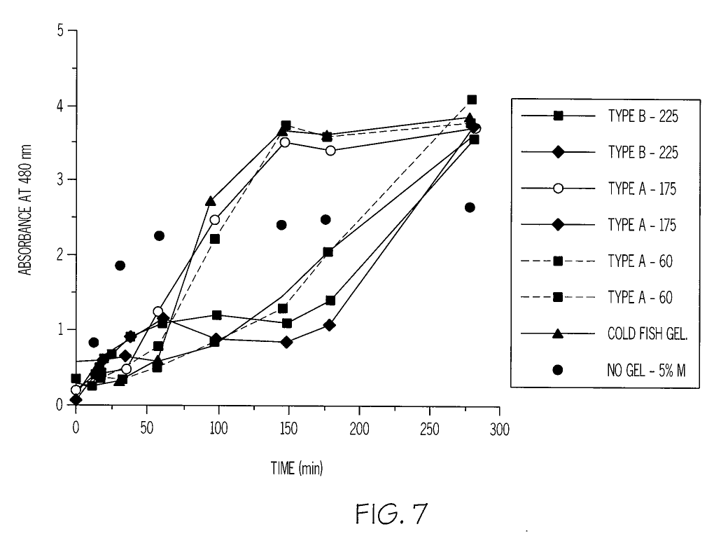

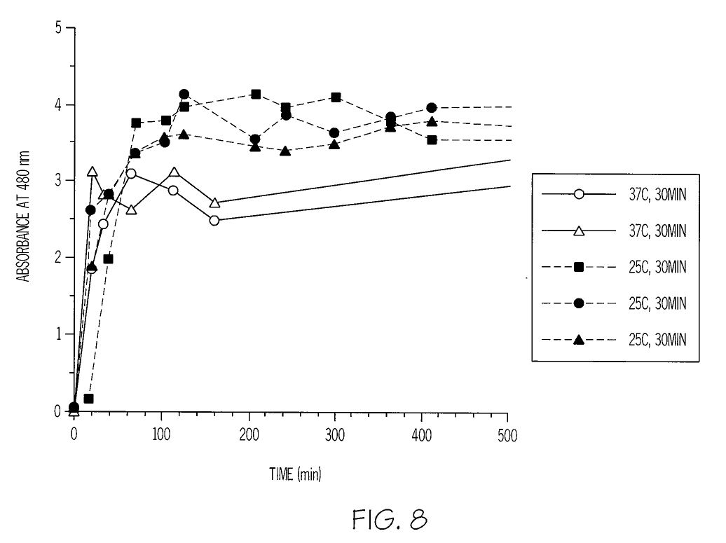

Next, the effect of varying gelatin type on the rate of color formation of transmissive sensors is disclosed. For the results shown in FIG. 7, a first layer of the sensors contained 25% ferric sulfate, 5% mannitol, and 5% of different types of gelatin (type is specified in legend). In particular, three types of gelatin were used: one from bovine skin, denoted as type B; one from porcine skin, denoted as type A; and one from cold-water fish skin. In addition, gelatins of different bloom numbers were used. For the second layer, 50% tetrabutylammonium thiocyanate in dichloromethane (w/v) was used. The sensors were placed in 58% relative humidity, and the rate of ferric thiocyanate production was measured spectrophotometrically at 480 nm for 5 hours. All sensors were cured at 4° C. for 64 hours before use.

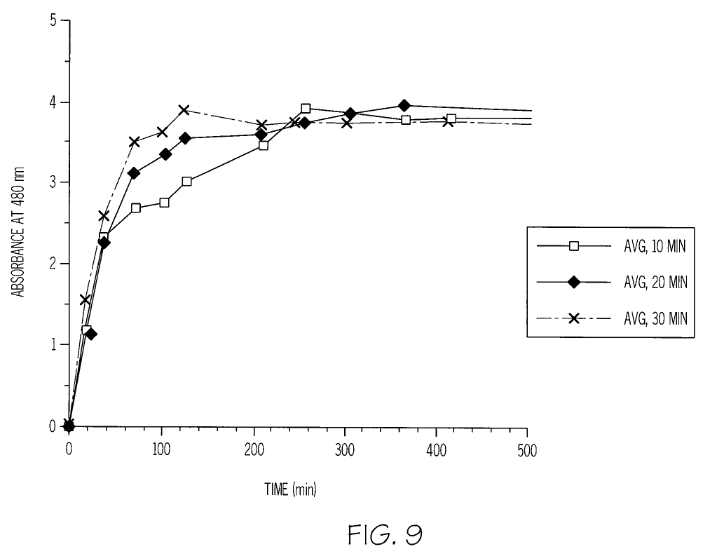

In an eight explicit embodiment, the effect of the drying step on transmissive sensors is disclosed. In an attempt to improve reproducibility, two separate studies were done. One used different temperatures to dry the iron layer and the other different drying times. These results are shown in FIGS. 8 and 9, respectively.

FIG. 8 shows the effect of drying temperature on rate of color formation of transmissive sensors. Sensors containing 25% ferric sulfate, 5% mannitol and 5% gelatin (225 bloom, type B) in the first layer were dried for 30 minutes at either 25° C. or 37° C. before the second layer of 50% tetrabutylammonium thiocyanate in dichloromethane was added. These sensors were then placed in a 60% relative humidity environment, and color formation was measured spectrophotometrically at 480 nm for 8 hours. Initially, color formation was greater for the plates dried at the lower temperature (25° C.). The kinetics of color formation is very similar, however, and the final color is spectrophotometrically much the same between the two samples. The drying temperature had little effect.

FIG. 9 compares the rate of color formation on transmissive sensors dried at 25° C. for various minutes. In particular, sensors containing 25% ferric sulfate, 5% mannitol, and 5% gelatin (type B, 225 bloom) in the first layer were dried at 25° C. for either 10 min, 20 min, or 30 min before adding the second layer of 50% tetrabutylammonium thiocyanate in dichloromethane. The sensors were then placed in a 60% relative humidity environment and color formation measured spectrophotometrically at 480 nm for 8 hours. In the plot of FIG. 9, each data point represents the average of three sensors. From the data, it is concluded that drying time has no major effect on the rate of color formation. Hence, a drying time of 30 minutes should be used to ensure the complete drying of the sensors to avoid any premature reaction.

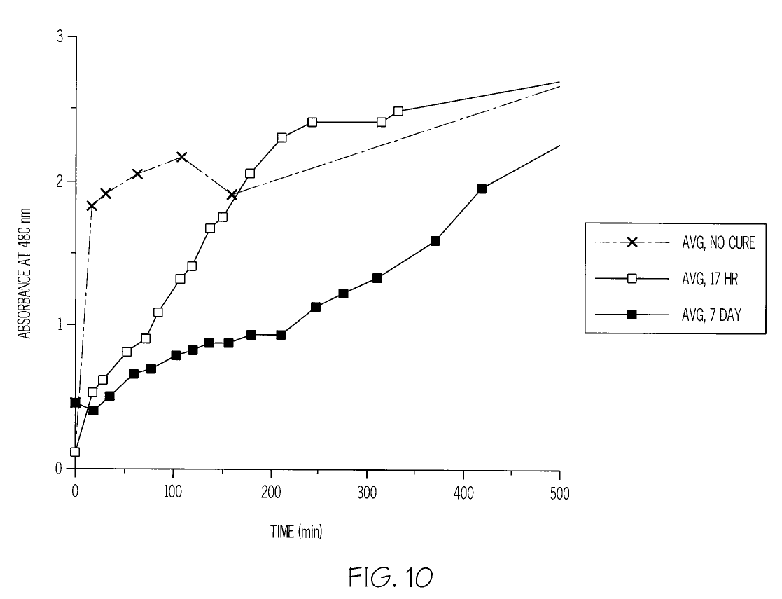

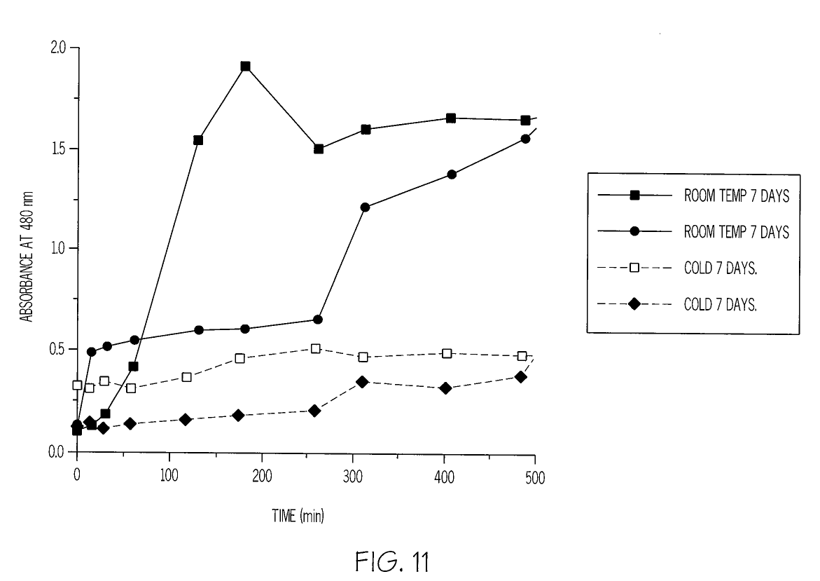

In a ninth explicit embodiment, a curing/aging phenomenon in gelatin-based sensors is disclosed. It was discovered that an aging or curing phenomenon occurred in sensors containing gelatin. Sensors stored for long times have slower reaction kinetics. Samples not containing gelatin did not exhibit this behavior however. This phenomenon is shown in more detail by comparing the reaction kinetics of sensors containing gelatin that were tested immediately after being made, to identical sensors that had been stored overnight, and to those that had been stored for one week. This comparison is shown in FIG. 10, which is a plot showing the effect of storage time on the rate of color formation of transmissive sensors (average absorbance values from triplicate readings).

For the data shown in FIG. 10, the sensors used a first layer of 25% ferric sulfate, 5% mannitol, and 5% gelatin, which was dried at 37° C. for 30 minutes. After varying times of storage, the second layer of 10 μl of 50% tetrabutylammonium thiocyanate in dichloromethane was applied and dried for 15 min. at 37° C. Storage times were within one hour, after 17 hours, or 7 days. The sensors were put in 60% relative humidity environment and the color change was followed at 480 nm.

To determine whether storage temperature affected curing, one set of sensors was stored for seven days at room temperature and another set was stored for 7 days at 4° C. The same storage protocol was utilized as was described earlier in the ninth explicit embodiment. Both sets of samples contained 25% ferric sulfate, 5% mannitol and 5% gelatin, and 50% tetrabutylammonium thiocyanate in dichloromethane. Sensors were removed, placed in 60% relative humidity environment and the color formation monitored at 480 nm. The results of this test are shown in FIG. 11.

The results suggest a storage temperature effect, in that the kinetics of color formation for those plates stored at 4° C. was slower than those that were stored at room temperature. This can be partially explained by the fact that gelatin forms more cross links when placed in a cold environment. This cross-linking causes a stronger film to be formed which can act to block external moisture absorption. Accordingly, sensors containing gelatin should be stored for a minimum of one week before use to reduce the effects of “curing time.”

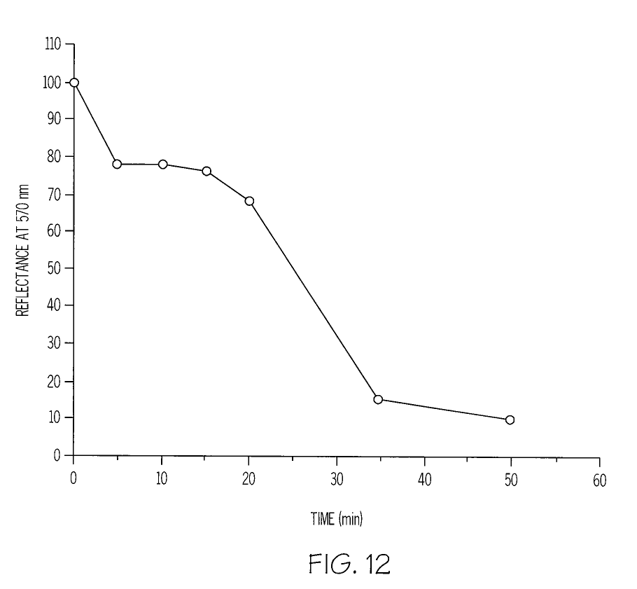

In a tenth explicit embodiment, a sodium hydroxide (NaOH)—cresol red reflective sensor is disclosed. In this embodiment of an acid-base indicator moisture sensor, calcium chloride is used as the hygroscopic material, cresol red as the acid-base indicator, and sodium hydroxide as the base. This sensor was formed from two pieces of pre-soaked absorbent paper which change from white to deep purple in humid conditions. One of the pieces of paper was soaked in an aqueous solution containing 75% calcium chloride and 1 mg/ml cresol red. The other piece was soaked in a 5% sodium hydroxide solution. Both pieces were air dried in a desiccator. When the two pieces were brought out in room humidity (about 45% RET) and placed in contact with each other (by calendering), specks of dark purple color began to appear and completely filled the one-inch diameter paper in about 45-50 minutes. The development of color was measured by reflectance at 570 nm. This data is shown in FIG. 12.

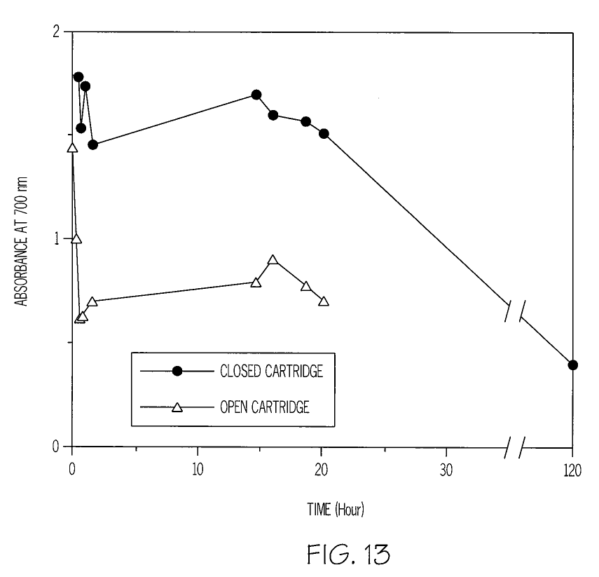

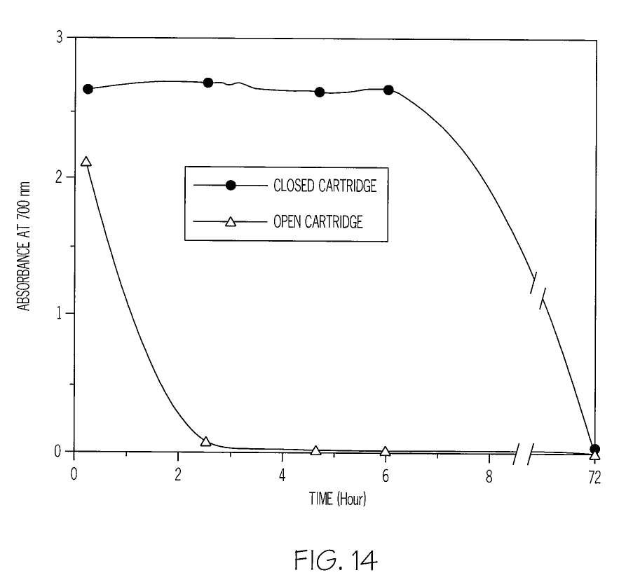

In an eleventh explicit embodiment, control of response by use of a diffusion channel is compared. In particular, FIGS. 13 and 14 show the effect of limiting access to atmospheric humidity by enclosure of transmissive sensors within a diagnostic assay device. Cobalt chloride films containing 50% cobalt chloride and 50% sucrose were placed in either the bottom half of a cartridge open to the atmosphere or in a sealed cartridges that connected to the atmosphere by a narrow capillary diffusion channel. For example, see U.S. Pat. No. D302,294 for the configuration of one type of cartridge, which is herein incorporated by reference. The larger capillary passageway (and thus the one which controlled diffusion) had a length of 2.0 cm, a height of 0.01 cm, and a width of 0.1 cm (cross sectional area of 0.1 mm2). The cartridges were exposed to either 34% relative humidity (FIG. 13) or 84% relative humidity (FIG. 14) and the absorbance measured at 700 nm over time (hours). The results demonstrate that the rate of response of a sensor can be controlled by enclosure of the sensor within a cartridge with capillary access to the environment.

The invention now being fully described, it will be apparent to one of ordinary skill in the art that many changes and modifications can be made thereto without departing from the spirit or scope of the appended claims.

1. A moisture sensor for detecting a cumulative exposure to moisture in an ambient environment, said moisture sensor comprising:
a matrix comprising:
a first reagent located in said matrix;
a second reagent located in said matrix, said first and second reagents interact only in the presence of moisture to produce a detectable, irreversible change in said matrix to indicate a level of cumulative moisture exposure; and
a hygroscopic material capable of absorption of the moisture from the ambient environment, wherein absorption of the moisture by said hygroscopic material causes the interaction of said first and second reagents which produces said detectable, irreversible change in said matrix.

2. The sensor of claim 1 wherein said first reagent is an enzyme and said second reagent is a chromogenic substrate of the enzyme.

3. The sensor of claim 1 wherein said first reagent that is an oxalic acid and said second reagent is a color change indicator.

4. The sensor of claim 1 wherein said first reagent that is a lactic acid and said second reagent is a color change indicator.

5. The sensor of claim 1 wherein said first reagent is an iron (III) salt and said second reagent is a thiocyanate salt.

6. The sensor of claim 1 wherein said first reagent is an iron (III) salt and said second reagent is a thiocyanate salt, and said hygroscopic material is said first reagent.

7. The sensor of claim 1, wherein said matrix comprises at least two layers and said first reagent and said second reagent are in different layers.

8. The sensor of claim 1, wherein said first reagent is selected from the group consisting of ferric chloride, ferric sulfate, and ferric nitrate and said second reagent is selected from the group consisting of ammonium thiocyanate, lithium thiocyanate, sodium thiocyanate, potassium thiocyanate, guanidine thiocyanate, and tetrabutylammonium thiocyanate.

9. The sensor of claim 1, wherein said first reagent is ferric sulfate and said second reagent is tetrabutylammonium thiocyanate.

10. The sensor of claim 1, wherein said matrix further comprises a film-forming agent.

11. The sensor of claim 1, wherein said matrix further comprises a glass-forming agent.

12. The sensor of claim 1, wherein said matrix further comprises a forming agent, said forming agent comprises one or more compounds selected from the group consisting of sucrose, gelatin, mannitol, trehalose, and PVP-10.

13. The sensor of claim 1, wherein said sensor further comprises a rate-controlling substance, wherein the rate of the interaction between said first and second reagents is controlled by said rate-controlling substance.

14. The sensor of claim 1, wherein said sensor further comprises a diffusion channel which exposes said hygroscopic material to the ambient environment, wherein said diffusion is shaped to control the rate of the interaction between said first and second reagents.

15. The sensor of claim 1, wherein said matrix further comprises a support.

16. The sensor of claim 1, wherein said matrix further comprises a support selected from the group consisting of paper, cloth, plastic, and glass.

17. The sensor of claim 1, wherein said change is detectable by an optical sensor.

18. The sensor of claim 1, wherein said change is a change in the optical transmittance of said matrix.

19. The sensor of claim 1, wherein said change is a change in the optical reflectance of said matrix.

20. The sensor of claim 1, wherein said change is an increase in opacity.

21. The sensor of claim 1, wherein said hygroscopic material is selected from the group consisting of calcium chloride, ferric chloride, sodium hydroxide, cobalt chloride, zinc chloride, and zinc bromide.

22. The sensor of claim 1, wherein said sensor is provided to a disposable diagnostic device containing analytical reagents sensitive to a known cumulative moisture exposure level and wherein said change is a color change at a cumulative moisture exposure level less than that of said known cumulative moisture exposure level.

23. A method for preventing the use of a disposable diagnostic device which has been exposed to excessive cumulative exposure to moisture in an ambient environment, said method comprising:
providing a moisture sensor to said disposable diagnostic device, wherein said moisture sensor comprises:
a layered matrix having:
a first reagent;
a second reagent, wherein said first and second reagents are capable of interacting to produce a detectable, irreversible change in said matrix; and
a hygroscopic material capable of absorption of moisture from the ambient environment, wherein absorption of the moisture by said hygroscopic material causes the interaction of said first and second reagents thereby producing said detectable, irreversible change in said matrix;
reading the cumulative moisture exposure indicated by said change in said matrix; and
excluding from analysis any of said devices having a cumulative moisture exposure in excess of a predetermined cumulative moisture exposure level.
